(19) United States
(12) Patent Application Publication
(10) Pub. No.: US 2006/0024711 A1
(43) Pub. Date: Feb. 2, 2006

(54) Methods for nucleic acid amplification and sequence determination

(75) Inventors: Stanley N. Lapidus, Bedford, NH (US); Philip R. Buzby, Brockton, MA (US)

Correspondence Address: SULLIVAN & WORCESTER LLP, ONE POST OFFICE SQUARE, BOSTON, MA, 02109, US

(73) Assignee: Helicos Biosciences Corporation, Cambridge, MA (US)

(21) Appl. No.: 11/167,046

(22) Filed: Jun. 23, 2005

Related U.S. Application Data
(60) Provisional application No. 60/585,565, filed on Jul. 2, 2004.

(51) Int. Cl. C12Q 1/68; C12P 19/34

(52) U.S. Cl. 435/6; 435/91.2

(57) ABSTRACT

The invention provides methods for sequencing a nucleic acid comprising conducting rolling circle amplification on a circular nucleic acid template, wherein the resulting amplicon is optionally anchored to a substrate in an individually optically resolvable manner, and performing a sequencing reaction.

### CROSS-REFERENCE TO RELATED APPLICATION

This application claims the benefit of U.S. Provisional Application No. 60/585,565, filed on Jul. 2, 2004, which is incorporated herein by reference.

### TECHNICAL FIELD OF THE INVENTION

The invention relates to methods and devices for sequencing a nucleic acid, and more particularly, to methods and devices for preparing a nucleic acid template for high throughput single molecule sequencing.

### BACKGROUND OF THE INVENTION

The completion of a consensus human genome sequence has given rise to inquiry into genetic differences within and between individuals as the basis for differences in biological function and dysfunction. For example, single nucleotide differences between individuals that give rise to single nucleotide polymorphisms (SNPs) can result in dramatic phenotypic differences. Those differences can be manifested in outward expressions of altered phenotype, can determine the likelihood that an individual will get a certain disease, or can determine how an individual will respond to a particular treatment. For example, most cancers develop from a series of genomic changes, some subtle and some major, that occur in a small subpopulation of cells. Knowledge of the sequence variations that lead to cancer will lead to an understanding of the etiology of the disease, as well as ways to treat and prevent it. An essential first step in understanding the genomic complexity of cancer and other diseases, as well as normal phenotypes and functions, is the ability to perform rapid high-resolution nucleic acid sequencing.

Conventional approaches to nucleic acid sequencing require the bulk preparation and analysis of nucleic acid. One common way to conduct bulk sequencing is by chain termination and gel separation, essentially as described in Sanger et al. (1997) Proc. Natl. Acad. Sci. USA, 74(12): 5463-67. The Sanger method requires the generation of a mixed population of nucleic acid fragments representing chain terminations at each base in a sequence. The fragments are then run on an electrophoretic gel and the nucleic acid sequence is obtained by determining the order of fragments in the gel. Another conventional bulk sequencing method involves the chemical degradation of nucleic acid fragments, for example, as described in Maxam et al. (1977) Proc. Natl. Acad. Sci. USA. 74: 560-64. Another bulk nucleic acid method involves sequencing by hybridization, for example, as described in Drmanac, et al. (1998) Nature Biotech., 16: 54-58, among others.

Numerous techniques and agents have been developed to improve the speed and fidelity of bulk nucleic acid sequencing. For example, the use of automated gel readers and improved polymerase enzymes have simplified and improved the efficiency of nucleic acid sequencing. However, those improvements are useful primarily in bulk sequencing methods and ensemble averaging, which lack single molecule resolution.

The focus of nucleic acid sequencing has shifted to the detection of genetic variation in individuals, in particular, the detection of variations that are associated with disease. Single molecule nucleic acid sequencing methods provide an alternative approach to bulk sequencing and can provide a more direct view of molecular activity without the need to infer process or function from ensemble averaging of data. While single molecule techniques have opened up new avenues for obtaining information on how changes in molecular structure affect functional variability, adequate resolution has been a problem due to the high background that is typical of fluorescence based sequencing assays. A need therefore exists for more effective and efficient methods and devices for single molecule nucleic acid sequencing, including innovations in template preparation, to improve nucleotide incorporation and signal detection.

### SUMMARY OF THE INVENTION

The invention provides methods for determining a nucleic acid sequence. In particular, the invention provides optical sequencing methods comprising amplification of a nucleic acid template by rolling circle amplification. In a preferred embodiment, rolling circle amplification produces an amplicon comprising a limited number of concatamers. The result is that an optical signal associated with an incorporated nucleotide is enhanced over background. For example, in one method according to the invention, rolling circle amplification produces an amplicon having not more than about one hundred linked complements of the nucleic acid template. The amplicon is attached to a substrate and a template-dependent sequencing-by-synthesis reaction is conducted on the limited multiple copies of the template.

According to the invention, a single stranded nucleic acid template (or a plurality of templates) is amplified using rolling circle amplification to produce linked copies of the complement of the original template. The nucleic acid template may be naturally circular or provided in a circular form, e.g., a DNA library, or may be circularized by any number of methods for circularizing single or double stranded nucleic acids. In one embodiment, the 5′ and 3′ ends of a single stranded nucleic acid are ligated, thereby circularizing the linear nucleic acid template. In another embodiment, nucleic acid linkers are first ligated to the 5′ and 3′ ends of a double stranded nucleic acid template, and the linkers are ligated, thereby circularizing the linear double stranded nucleic acid template. The double stranded circular template is then denatured so that a rolling circle amplification primer can be annealed to one of the single template strands. The primer hybridization site preferably spans the ligation site, such that the primer does not hybridize, or hybridized less efficiently, to the linear nucleic acid template.

In one preferred embodiment, single molecule sequence is conducted on the amplified concatamers. The amplicon is anchored to a substrate such that at least some of them are individually optically resolvable with respect to other amplicons. Because an amplicon comprises a plurality of identical complements of the template, nucleotide incorporation occurs at multiple identical loci during each step of the sequencing reaction. Thus, within each individual optical field, the fluorescence from multiple identical loci is optically detectable, thereby providing a signal that is boosted relative to that produced by a single incorporation on a single template/primer duplex. In this respect, the invention comprises a combination of limited template amplification and attachment to a substrate in an individually optically resolvable position in order to boost detectable incorporation signal in a template-dependent sequencing-by-synthesis reaction.

Methods according to the present invention comprise circularizing at least one nucleic acid template of interest and exposing the circularized template(s) to a primer, a polymerizing agent, and labeled nucleotides in order to conduct rolling circle amplification. While rolling circle amplification produces generally fewer amplicons than PCR, it still can result in the generation of many thousands of copies of the template. Methods of the invention limit amplification cycles as compared to traditional rolling circle amplification, to produce about two to about one hundred linked complementary copies of the circularized template. In some embodiments, amplicon(s) of about two to about fifty complements, about two to about twenty complements, or preferably about two to about eight complements are produced. In certain embodiments the number of cycles of amplification is limited by limiting the amount of nucleotides in the reaction mixture. In other embodiments, the number of cycles of amplification is limited by inactivating the polymerase after about two to about one hundred cycles. Other methods for limiting the rate or extent of amplification are known in the art.

Methods according to the invention also comprise anchoring the amplicon(s) to a substrate. In certain embodiments, the rolling circle amplification primer is an oligonucleotide, a portion of which is anchored to the substrate so that the template hybridizes to the anchored primer and extension of the primer on the template creates an anchored amplicon. In other embodiments the amplification is conducted in solution and, following the reaction, the resulting amplicon is anchored to the substrate using any mode of attachment. Preferred surfaces for oligonucleotide attachment include, but are not limited to, epoxides, silanes, glass, polyelectrolyte multilayers, and derivatives of the foregoing. Examples of preferred modes of attachment of a concatameric duplex to a surface include, but are not limited to, direct amine attachment, attachment via a binding pair, such as biotin/streptavidin, dintrophenol/anti-dinitrophenol, digoxigenin/anti-digoxigenin, and other antigen/antibody or receptor binding pairs.

Sequencing according to the invention comprises template-dependent nucleic acid synthesis. In a preferred embodiment, nucleic acid sequencing primers are exposed to amplicons having at least one primer binding site. A polymerase then directs the extension of the primer(s) in a template-dependent fashion in the presence of labeled nucleotides or nucleotide analogs. According to one aspect of the invention, amplicons are support-bound in a manner that allows unique optical identification of signaling events from the labeled nucleotide or nucleotide analogs as they are incorporated into the growing primer strand.

Preferred methods of the invention comprise optically detecting incorporation of a nucleotide or nucleotide analog in a template-dependent primer extension reaction. In preferred embodiments, nucleotides are labeled for detection, preferably with a fluorescent label. In one embodiment, methods of the invention comprise detecting coincident fluorescence emission from at least two labeled nucleotides incorporated at the same loci on different copies of the template within the same amplicon.

Labeled nucleotides of the invention include any nucleotide that has been modified to include a label that is directly or indirectly detectable. Such labels include optically-detectable labels such fluorescent labels, including fluorescein, rhodamine, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY, ALEXA, or a derivative or modification of any of the foregoing. In one embodiment of the invention, fluorescence resonance energy transfer (FRET) technology is employed to produce a detectable, but quenchable, label. FRET may be used in the invention by modifying the primer to include a FRET donor moiety and using nucleotides labeled with a FRET acceptor moiety.

Methods of the invention address the problem of reduced detection due to a failure of some strands in a given cycle to incorporate labeled nucleotide. In each incorporation cycle, a certain number of strands fail to incorporate a nucleotide that should be incorporated based upon their ability to hybridize to a nucleotide present in the template. In a preferred embodiment, the amplicon provides a benefit of bulk sequencing to a single molecule sequencing reaction, such that each complement in an amplicon need not incorporate a labeled nucleotide or nucleotide analog in every incorporation cycle. Incorporation of a labeled nucleotide at one or more independent loci in an amplicon provides a detectable signal. In certain embodiments, a low concentration of unlabeled nucleotides is added with the labeled nucleotides or nucleotide analogs. In other embodiments, after removing unbound labeled nucleotide, the sample is exposed to unlabeled nucleotide, preferably in excess, of the same species. In either situation, the unlabeled nucleotide “fills in” the positions in which hybridization of the labeled nucleotide did not occur.

The invention is useful in sequencing any form of nucleic acid, such as double-stranded DNA, single-stranded DNA, single-stranded DNA hairpins, DNA/RNA hybrids, RNAs with a recognition site for binding of the polymerizing agent, and RNA hairpins, for example. The invention is particularly useful in creating amplicons for use as templates for high throughput sequencing of single molecule nucleic acids in which a plurality of amplicons are attached to a solid support in a spatial arrangement such that each amplicon is individually optically resolvable. According to the invention, each detected incorporated label represents a single polynucleotide.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 shows a sample method of preparing a nucleic acid template for circularization.

FIG. 2A shows a schematic of a template circularized by annealing to an anchor primer.

FIG. 2B shows a collection of the complexes in FIG. 2A hybridized to a surface of a substrate.

FIG. 3A shows nucleic acid template ligation reactions of oligonucleotides of varying lengths, in the presence (+) or absence (−) of CircLigase™ enzyme.

FIG. 3B shows the same reactions as those shown in FIG. 3A with the addition of Exo I enzyme.

FIG. 4 shows rolling circle amplification reactions using primer A or primer B and circular template A or circular template B, alone and in various combinations. Lane 1 is a marker lane; Lane 2 reaction has primer A and circular template A, with polymerase; Lane 3 reaction has primer A and circular template A, without polymerase; Lane 4 reaction has primer A and linear template A, with polymerase; Lane 5 reaction has primer B and circular template A, with polymerase; Lane 6 reaction has primer A and circular template B, with polymerase; Lane 7 reaction has primer B and circular template B, with polymerase; Lane 8 reaction has primer B and circular template B, with polymerase; Lane 9 reaction has primer B and linear template B, with polymerase; Lane 10 reaction has primer A only, with polymerase; Lane 11 reaction has circular template A only, with polymerase; Lane 12 reaction has linear template A only, with polymerase; Lane 13 reaction has circular template B only, with polymerase; Lane 14 reaction has linear template B only, with polymerase; Lane 15 reaction has markers; Lane 16 reaction has primer B only, with polymerase; Lane 16 reaction has water only, with polymerase.

FIG. 5 shows the results of rolling circle amplification reactions using either a 53 base oligonucleotide or a 66 base oligonucleotide in the presence (+) or absence (−) of CircLigase™ enzyme, and in the presence of various amounts of polyethylene glycol (PEG).

FIG. 6 shows incorporations of fluorescently labeled nucleotides at multiple identical loci on 3 different amplicons. FIG. 6 also shows unlabeled nucleotides “filling-in” on some strands.

FIG. 7 shows a schematic of a detection result. Individual molecules are optically resolvable, however, the close-up shows that multiple labeled nucleotides provide a combined signal at one position on the array.

FIG. 8A shows an electropherogram of the sequencing of a 53 nucleotide circularized template DNA.

FIG. 8B shows the nucleotide sequence of 5 repeats of the template DNA in the rolling circle amplified product. The sequence that is complementary to the primer sequence is bolded.

FIG. 9 shows schematically the optical set-up of a detection system for total internal reflection microscopy.

A detailed description of embodiments of the invention is provided below. Other embodiments of the invention are apparent upon review of the detailed description that follows.

### DETAILED DESCRIPTION OF THE INVENTION

The invention provides methods for determining a sequence of a nucleic acid. Methods according to the invention encompass the preparation of template nucleic acids that provide improve nucleotide incorporation and signal detection in sequencing reactions. Methods of the invention also are useful for overcoming obstacles to single molecule sequencing, including, for example, low extension yield due to difficulty in incorporating labeled nucleotides and detecting signal over accumulated background.

The invention relates to the use of rolling circle amplification for the amplification of nucleic acid sequencing template to improve signal detection. Rolling circle amplification is a method of generating multiple linear copies (concatamers), linked end-to-end, of a circular nucleic acid template. In vivo, bacterial plasmids and some viruses replicate by rolling circle amplification by recruiting host DNA replication proteins, autonomously synthesizing other necessary proteins, and initiating replication by nicking one of the two strands. The replication machinery synthesizes a complementary strand to the remaining circular template, and the self-proteins cleave and circularize the complementary strand replication products into new plasmids. See e.g., Khan (1997) Microb. Molec. Biol. Rev., 61(4): 442-55.

Methods of the invention comprise amplifying a nucleic acid template to create an amplicon comprising concatamerized complements of the template, wherein the amplicon is anchored to a substrate and the sequence of at least a portion of the template is determined. Preferred methods comprise conducting a limited number of cycles of rolling circle amplification to produce an amplicon comprising a plurality of complements of the template that are individually optically resolvable from other sets of linked templates. When functioning as a sequencing template, an amplicon comprising a plurality of identical complements of the nucleic acid template facilitates simultaneous nucleotide incorporation at multiple identical loci during each cycle of the sequencing reaction.

Methods of the invention provide improvements on the ability to incorporate labeled nucleotides and the ability to detect incorporation events during sequencing. In particular, methods of the invention are useful in a single molecule sequencing system employing fluorescently labeled nucleotides, in which accumulation of fluorescent background typically makes signal detection challenging.

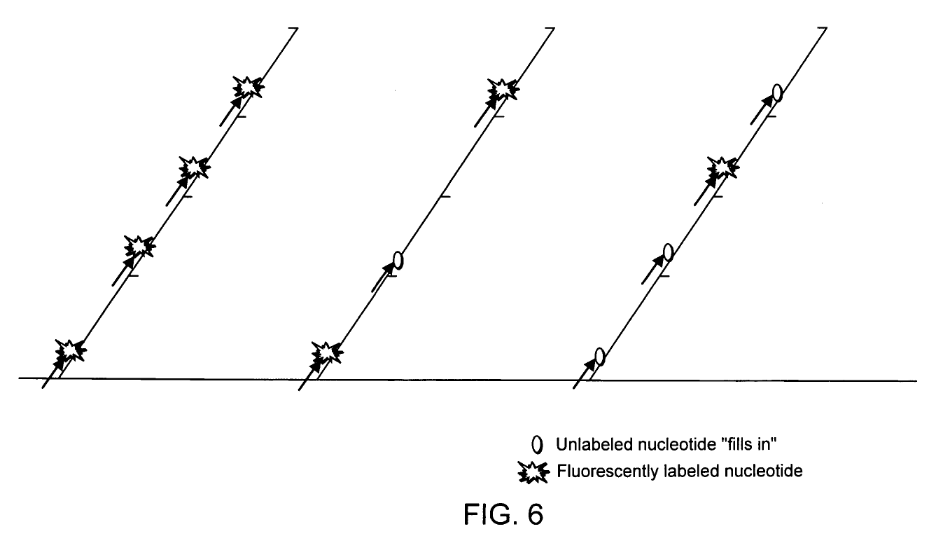

In a preferred embodiment, an amplicon is exposed to a sequencing primer, a polymerase, and a labeled nucleotide, and, as shown in FIG. 6, a plurality of sequencing primers anneal to one or more complements of an amplicon. The annealing of multiple primers initiates at least one sequencing reaction per amplicon, and incorporates labeled nucleotides downstream of each primer. Preferably, the simultaneous nucleotide incorporations at a plurality of identical loci create an aggregate fluorescent signal that is detectable over accumulated background fluorescence on the reaction substrate.

The present invention comprises embodiments wherein rolling circle amplification is conducted such that the primer is anchored to a substrate and hybridizes to a template prior to amplification. In another embodiment, amplification takes place prior to hybridization of the primer to the substrate. In an embodiment, the primer sequence comprises the complement of at least a portion of both ends of the linear template such that the primer only anneals, or anneals more efficiently with, the circular template. Optionally, the amplification primers are anchored to the substrate in a manner that makes the resulting amplicons individually optically resolvable from one another. Methods of the invention also comprise embodiments wherein the rolling circle amplification is conducted in solution and amplicons are subsequently anchored to the surface of the substrate.

Accordingly, an aspect of the invention is the ability to facilitate detection of coincident fluorescence emission from at least two labeled nucleotides incorporated at the same loci on different complements of a template within the same amplicon. Additional aspects of the invention are described in the following sections and illustrated by the Examples.

Methods according to the invention provide simple and accurate sequencing with further applications in disease detection and diagnosis and individual genome analysis. Methods according to the invention provide de novo sequencing, sequence analysis, DNA fingerprinting, polymorphism identification, for example single nucleotide polymorphism (SNP) detection, as well as applications in cancer diagnosis and therapeutic treatment selection. Applied to RNA sequences, methods according to the invention identify alternate splice sites, enumerate copy number, measure gene expression, identify unknown RNA molecules present in cells at low copy number, annotate genomes by determining which sequences are actually transcribed, determine phylogenic relationships, elucidate differentiation of cells, and facilitate tissue engineering. Methods according to the invention also can be used to analyze activities of other biomacromolecules such as RNA translation and protein assembly.

Certain aspects of the invention lead to more sensitive detection of incorporated signals and faster sequencing. Methods of the invention include amplifying the nucleic acid template by conducting rolling circle amplification. Methods of the invention also include detecting incorporation of the nucleotide or nucleotide analog in the growing primer strand and, repeating the determining step to determine a sequence of the nucleic acid template. By creating a complementary sequence to the template in the rolling circle amplification step, the sequence of the template can be directly compiled during the determining step based upon sequential incorporation of the nucleotides into the primer.

Many methods are available for the isolation and purification of nucleic acid templates for use in the present invention. Preferably, the target molecules or nucleic acids are sufficiently free of proteins and any other interfering substances to allow target-specific primer annealing and extension. Preferred purification methods include (i) organic extraction followed by ethanol precipitation, e.g., using a phenol/chloroform organic reagent, preferably using an automated DNA extractor, e.g., a Model 341 DNA Extractor available from PE Applied Biosystems (Foster City, Calif.); (ii) solid phase adsorption methods; and (iii) salt-induced DNA precipitation methods, such methods being typically referred to as “salting-out” methods. Optimally, each of the above purification methods is preceded by an enzyme digestion step to help eliminate protein from the sample, e.g., digestion with proteinase K or other like protease.

Methods of the invention require a circular nucleic acid template, however, the nucleic acid can come from a variety of sources. For example, nucleic acids can be naturally occurring DNA or RNA isolated from any source, recombinant molecules, cDNA, or synthetic analogs, as known in the art. The nucleic acid template may comprise genomic DNA, DNA fragments (e.g., such as exons, introns, regulatory elements, such as promoters, enhancers, initiation and termination regions, expression regulatory factors, expression controls, and other control regions), DNA comprising one or more single-nucleotide polymorphisms (SNPs), allelic variants, and mutant nucleic acid. The nucleic acid template may also be an RNA, such as mRNA, tRNA, rRNA, ribozymes, splice variants, antisense RNA, and RNAi, for example. Also contemplated as useful according to the invention are RNA with a recognition site for binding a polymerase, transcripts of a single cell, organelle or microorganism, and all or portions of RNA complements of one or more cells, for example, cells from different stages of development, differentiation, or disease, and cells from different species. Nucleic acids may be obtained from any nucleic acid source, such as a cell of a person, animal, or plant, or cellular or microbial organism, such as a bacteria, or other infectious agent, such as a virus. Individual nucleic acids may be isolated for analysis, for example, from single cells in a patient sample comprised of cancerous and precancerous cells.

In a preferred embodiment, the nucleic acid template is genomic DNA from one or more cells that is circularized using any method known in the art, including enzymatic or chemical circularization. Chemical methods employ known coupling agents such as BrCN plus imidazole and a divalent metal, N-cyanoimidazole with ZnCl₂, 1-(3-dimethylaminopropyl)-3 ethylcarbodiimide HCl, and other carbodiimides and carbonyl diimidazoles. The ends of a linear template may also be joined by condensing a 5′-phosphate and a 3′-hydroxyl, or a 5′-hydroxyl and a 3′-phosphate. DNA ligase or RNA ligase may be used to enzymatically join the two ends of a linear template, with or without an adapter molecule or linkers, to form a circle. For example, T4 RNA ligase couples single-stranded DNA or RNA, as described in D. C. Tessier et al. (1986) Anal. Biochem., 158: 171-78. CircLigase™ (Epicentre, Madison, Wis.) may also be used to catalyze the ligation of a single stranded nucleic acid. Alternatively, a double stranded E. coli or T4 DNA ligase may be used to join the 5′ and 3′ ends of a double stranded nucleic acid and the double stranded template denatured prior to annealing to the primer.

In some embodiments, templates are digested with a restriction enzyme to yield fragments of any size and then cloned or subcloned into a known vector. In one embodiment, nucleic acid templates, such as linear fragments of genomic DNA, are ligated to linker oligonucleotides. The linker/template complexes are denatured and exposed to a substrate comprised of anchored oligonucleotides. Linker sequences hybridize to the anchor oligonucleotides in a conformation such that the 5′ phosphate and 3′ hydroxyl of the linker/template complex are adjacent to each other. The 5′ and 3′ ends are then ligated, creating a circular molecule. In another embodiment, the linear template is circularized and ligated prior to annealing to the primer. By targeting the primer to the 5′ and/or 3′ ends of the linear template, the primer will be selective for circularized template.

Generally, nucleic acid templates may have a length of about 5 bases, about 10 bases, about 20 bases, about 30 bases, about 40 bases, about 50 bases, about 60 bases, about 70 bases, about 80 bases, about 90 bases, about 100 bases, about 200 bases, about 500 bases, about 1 kb, about 3 kb, about 10 kb, or about 20 kb and so on. Preferably, nucleic acid templates are about 10 to about 50 bases.

Methods according to the invention provide for conducting rolling circle amplification on a nucleic acid template. The amplification may be performed on a template that has been circularized by annealing to an anchor primer, before or after the anchor primer is hybridized to a substrate. Rolling circle replication requires effective amounts of reagents including a polymerase, nucleotides, a primer, and a template. Any polymerase capable of performing rolling circle amplification may be used in the reaction, for example, phi 29 DNA polymerase, Taq polymerase, T7 mutant DNA polymerase, T5 DNA polymerase, Klenow, Sequenase, other known DNA polymerases, RNA polymerases, thermostable polymerases, thermodegradable polymerases, and reverse transcriptases. See e.g., Blanco et al., U.S. Pat. Nos. 5,198,543 and 5,001,050; Doublie et al. (1998) Nature, 391:251-58; Ollis et al. (1985) Nature, 313: 762-66; Beese et al., (1993) Science 260: 352-55; Korolev et al.(1995) Proc. Natl. Acad. Sci. USA, 92: 9264-68; Keifer et al. (1997) Structure, 5:95-108; and Kim et al. (1995) Nature, 376:612-16.

A target nucleic acid may be immobilized or anchored on a substrate to prevent its release into surrounding solution or other medium. For example, an anchor primer, anchor primer/template complex, or amplicon may be anchored or immobilized by covalent bonding, non-covalent bonding, ionic bonding, hydrogen bonding, van der Waals forces, hydrophobic bonding, or a combination thereof. The anchoring or immobilizing of a molecule to the substrate may utilize one or more binding-pairs, including, but not limited to, an antigen-antibody binding pair, a streptavidin-biotin binding pair, photoactivated coupling molecules, digoxigenin/anti-digoxigenin, and a pair of complementary nucleic acids.

In some embodiments, single molecules of target nucleic acids are separately synthesized, and subsequently attached to a substrate for sequence determination and analysis. In these embodiments, the nucleic acid may be attached to the substrate through a covalent linkage or a non-covalent linkage. When the nucleic acid is attached to the substrate through a non-covalent linkage, the nucleic acid includes one member of specific binding pair, e.g., biotin, the other member of the pair being attached to the substrate, e.g., avidin or streptavidin. Several methods are available for covalently linking polynucleotides to substrates, e.g., through reaction of a 5′-amino polynucleotide with an isothiocyanate-functionalized glass support. A wide range of exemplary linking moieties for attaching primers onto solid supports either covalently or non-covalently are known in the art.

Depending on the template, a DNA polymerase, an RNA polymerase, a reverse transcriptase, or any enzyme capable of polymerizing a nucleic acid strand complementary to the nucleic acid template may be used in the primer extension reactions. Generally, the polymerase according to the invention has high incorporation accuracy and a processivity (number of nucleotides incorporated before the polymerase dissociates from the target nucleic acid) of at least about 20 nucleotides. Nucleotides may be selected to be compatible with the polymerase.

Methods of the invention comprise conducting primer extension reactions with target nucleic acids that are attached to a substrate, surface, support or an array. Each member of the plurality of target nucleic acids may be covalently attached to a surface including glass or fused silica. For example, each member of the plurality of target nucleic acids may be covalently attached to a surface that has reduced background fluorescence with respect to glass, polished glass, fused silica or plastic. Examples of surfaces appropriate for the invention include, for example, polytetrafluoroethylene or a derivative of polytetrafluoroethylene, such as silanized polytetrafluoroethylene, epoxides, derivatized epoxides, polyelectrolyte multilayers, and others.

In some embodiments, a primer, a target polynucleotide-primer complex, and/or a polymerase is bound or immobilized on the surface of the substrate or array. The surface to which oligonucleotides are attached may be chemically modified to promote attachment, improve spatial resolution, and/or reduce background. Exemplary substrate coatings include polyelectrolyte multilayers. Typically, these are made via alternate coatings with positive charge (e.g., polyllylamine) and negative charge (e.g., polyacrylic acid). Alternatively, the surface may be covalently modified, as with vapor phase coatings using 3-aminopropyltrimethoxysilane. In an embodiment, the primer attaches to the solid support by direct amine end attachment of the 3′ end of primer.

Solid supports of the invention may comprise glass, fused silica, epoxy, plastic, metal, nylon, gel matrix or composites. Furthermore, the substrate or support may include a semi-solid support (e.g., a gel or other matrix), and/or a porous support (e.g., a nylon membrane or other membrane). In an embodiment, the surface of the solid support is coated with epoxide. The surface of the substrate or support may be planar, curved, pointed, or any suitable two-dimensional or three-dimensional geometry. The invention also contemplates the use of beads or other non-fixed surfaces. Target molecules or nucleic acids may be synthesized on a substrate to form a substrate including regions coated with nucleic acids or primers, for example. In some embodiments, the substrate is uniformly comprised of nucleic acid targets or primers. That is, within each region in a substrate or array, the same nucleic acid or primer may be synthesized.

Analyzing a nucleic acid template sequence by sequencing its complement strand may involve hybridizing a primer to the amplicon product of rolling circle amplification. If part of the region downstream of the sequence to be analyzed is known, a specific primer may be constructed and hybridized to this region of the nucleic acid template. Alternatively, if sequences of the downstream region on the nucleic acid template are not known, universal or random primers may be used in random primer combinations. Alternatively, known sequences may be biotinylated and ligated to the targets. In yet another approach, a nucleic acid may be digested with a restriction endonuclease, and primers designed to hybridize with the known restriction sites that define the ends of the fragments produced.

Primers for both rolling circle amplification and sequencing may be synthetically made using conventional nucleic acid synthesis techniques. For example, primers may be synthesized on an automated DNA synthesizer, e.g. an Applied Biosystems, Inc. (Foster City, Calif.) model 392 or 394 DNA/RNA Synthesizer, using standard chemistries, such as phosphoramidite chemistry, and the like. Alternative chemistries, e.g., resulting in non-natural backbone groups, such as phosphorothioate and the like, may also be employed provided that, for example, the resulting oligonucleotides are compatible with the polymerizing agent. The primers may also be ordered commercially from a variety of companies that specialize in custom nucleic acids such as Operon Inc. (Alameda, Calif.).

In some instances, the sequencing primer includes a label. When hybridized to a linked nucleic acid molecule or amplicon, the label facilitates locating the bound molecule through imaging. For example, the primer is labeled with a fluorescent labeling moiety (e.g., Cy3 or Cy5), or any other means used to label nucleotides. The detectable label on the primer may be different from the label on the nucleotides or nucleotide analogs in the subsequent extension reactions. Suitable fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

Sequencing methods according to the invention include exposing a nucleic acid template to at least one nucleotide, labeled nucleotide, or nucleotide analog allowing for extension of the primer. A nucleotide or nucleotide analog includes any base or base-type including adenine, cytosine, guanine, uracil, or thymine bases. Additional nucleotide analogs include xanthine or hypoxanthine, 5-bromouracil, 2-aminopurine, deoxyinosine, or methylated cytosine, such as 5-methylcytosine, N4-methoxydeoxycytosine, and the like. Also included are bases of polynucleotide mimetics, such as methylated nucleic acids, e.g., 2′-O-methRNA, peptide nucleic acids, modified peptide nucleic acids, and any other structural moiety that acts substantially like a nucleotide or base, for example, by exhibiting base-complementarity with one or more bases that occur in DNA or RNA and/or being capable of base-complementary incorporation.

Labeled nucleotides for use in the invention are any nucleotide that has been modified to include a label that is directly or indirectly detectable. Preferred labels include optically-detectable labels, including fluorescent labels, such as fluorescein, rhodamine, derivatized rhodamine dyes, such as TAMRA, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY,120 ALEXA, or a derivative or modification of any of the foregoing. As the skilled artisan will appreciate, however, any detectable label may be used to advantage within the principles of the invention.

According to the invention, identification of nucleotides in a sequence may be accomplished using fluorescence resonance energy transfer (FRET). Generally, a FRET donor (e.g., cyanine-3) is placed on the primer, on the polymerase, or on a previously incorporated nucleotide. The primer/template complex then is exposed to a nucleotide comprising a FRET acceptor (e.g., cyanine-5). If the nucleotide is incorporated, the acceptor is activated and emits detectable radiation, while the donor goes dark.

The fluorescently labeled nucleotides may be obtained commercially (e.g., from NEN DuPont, Amersham, and BDL). Alternatively, fluorescently labeled nucleotides may also be produced by various techniques, such as those described in Kambara et al. (1988) Bio/Technol., 6:816-21; Smith et al. (1985) Nucl. Acid Res., 13: 2399-2412; and Smith et al.(1986) Nature, 321: 674-79. The fluorescent dye is preferably linked to the deoxyribose by a linker arm that is easily cleaved by chemical or enzymatic means. The length of the linker between the dye and the nucleotide can impact the incorporation rate and efficiency. See Zhu et al. (1997) Cytometry, 28: 206. There are numerous linkers and methods for attaching labels to nucleotides, as shown in Oligonucleotides and Analogues: A Practical Approach (1991) (IRL Press, Oxford); Zuckerman et al. (1987) Polynucleotides Res., 15: 5305-21; Sharma et al. (1991) Polynucleotides Res., 19: 3019; Giusti et al. (1993) PCR Methods and Applications, 2: 223-27; Fung et al., U.S. Pat. No. 4,757,141; Stabinsky, U.S. Pat. No. 4,739,044; Agrawal et al. (1990) Tetrahedron Letters, 31: 1543-46; Sproat et al. (1987), Polynucleotides Res., 15: 4837; and Nelson et al. (1989) Polynucleotides Res., 17: 7187-94. Extensive guidance exists in the literature for derivatizing fluorophore and quencher molecules for covalent attachment via common reactive groups that may be added to a nucleotide. Many linking moieties and methods for attaching fluorophore moieties to nucleotides also exist, as described in Oligonucleotides and Analogues, supra; Guisti et al., supra; Agrawal et al, supra; and Sproat et al., supra.

While the invention is exemplified herein with fluorescent labels, the invention is not so limited and may be practiced using nucleotides labeled with any form of detectable label, including radioactive labels, chemoluminescent labels, luminescent labels, phosphorescent labels, fluorescence polarization labels, and charge labels.

The sequencing primer may be hybridized to the amplicon before or after the amplicon is attached on a surface of a substrate or array. Primer annealing is performed under conditions that are stringent enough to require sufficient sequence specificity, yet permissive enough to allow formation of stable hybrids at an acceptable rate. The temperature and time required for primer annealing depend upon several factors including nucleotide composition, nucleic acid length, and the concentration of the primer; the nature of the solvent used, for example, the concentration of DMSO, polyethylene glycol (PEG), formamide, or glycerol; as well as the concentrations of counter ions, such as magnesium and manganese. Typically, hybridization with synthetic polynucleotides is carried out at a temperature that is approximately 5° C. to approximately 10° C. below the melting temperature (Tm) of the target polynucleotide-primer complex in the annealing solvent.

After creating the amplicon and linking it on a substrate, primer extension reactions may be performed to analyze the sequence of the nucleic acid template sequence by synthesizing a complement to the amplicon. The primer is extended by a polymerase in the presence of a nucleotide or nucleotide analog bearing a detectable label at a temperature of about 10° C. to about 70° C., about 20° C. to about 60° C., about 30° C. to about 50° C., or preferably at about 37° C. In other embodiments, two, three or all four types of nucleotides are present, each bearing a detectably distinguishable label. In some embodiments of the invention, a combination of labeled and non-labeled nucleotides or nucleotide analogs is used in the primer extension reaction for analysis.

Any detection method may be used that is suitable for the type of label employed. Thus, exemplary detection methods include radioactive detection, optical absorbance detection, such as UV-visible absorbance detection, and optical emission detection, such as fluorescence or chemiluminescence detection. For example, extended primers may be detected on a substrate by scanning all or portions of each substrate simultaneously or serially, depending on the scanning method used. For fluorescence labeling, selected regions on a substrate may be serially scanned one-by-one or row-by-row using a fluorescence microscope. Hybridization patterns may also be scanned using a CCD camera (e.g., Model TE/CCD512SF, Princeton Instruments, Trenton, N.J.) with suitable optics, such as total internal reflection optics, or may be imaged by TV monitoring. To detect radioactive signals, a phosphorimager device may be used. Other commercial suppliers of imaging instruments include General Scanning Inc. (Watertown, Mass.), Genix Technologies (Waterloo, Ontario, Canada), and Applied Precision Inc. Such detection methods are particularly useful to achieve simultaneous scanning of multiple tag complement regions. As such, embodiments of the present invention provide for detection of a single nucleotide into a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode may be used. Full field imaging with a two-stage image intensified COD camera also may be used. Additionally, low noise cooled CCD may also be used to detect single fluorescent molecules.

The detection system for the signal may depend upon the labeling moiety used, which is defined by the chemistry available. For optical signals, a combination of an optical fiber or charged couple device (CCD) may be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems may be used. Various physical orientations for the detection system are available and discussion of important design parameters is provided in the art.

A number of approaches may be used to detect incorporation of fluorescently-labeled nucleotides into a single polynucleotide molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. It is sometimes referred to as a high-efficiency photon detection system. Suitable photon detection systems include, but are not limited to, photodiodes and intensified CCD cameras. For example, an intensified charge couple device (ICCD) camera may be used. The use of an ICCD camera to image individual fluorescent dye molecules in a fluid near a surface provides numerous advantages. For example, with an ICCD optical setup, it is possible to acquire a sequence of images (movies) of fluorophores.

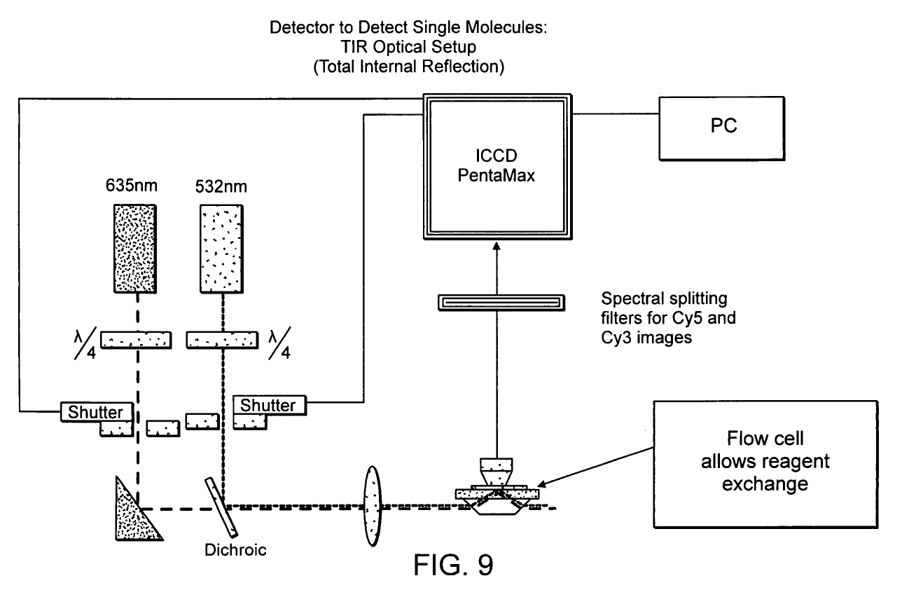

Some embodiments of the present invention use total internal reflection fluorescence (TIRF) microscopy for two-dimensional imaging, as shown in FIG. 9. Total internal reflection microscopy uses totally internally reflected excitation light and is well known in the art. In certain embodiments, detection is carried out using evanescent wave illumination and total internal reflection fluorescence microscopy. An evanescent light field may be set up at the surface, for example, to image fluorescently-labeled polynucleotide molecules. When a laser beam is totally reflected at the interface between a liquid and a solid substrate (e.g., a glass), the excitation light beam penetrates only a short distance into the liquid. In other words, the optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the immobilized target polynucleotide-primer complex in the presence of a polymerase. TIRF microscopy may then be used to visualize the immobilized target polynucleotide-primer complex and/or the incorporated nucleotides with single molecule resolution. With TIRF technology, the excitation light (e.g., a laser beam) illuminates only a small volume of solution close to the substrate, called the excitation zone. Signals from free (i.e., unincorporated) nucleotides in solution outside the excitation zone would not be detected. Signals from free nucleotides that diffuse into the excitation zone would appear as a broad band background because the free nucleotides move quickly across the excitation zone.

TIRF microscopy has been used to examine various molecular or cellular activities. TIRF examination of cell/surface contacts dramatically reduces background from surface autofluorescence and debris. TIRF also has been combined with fluorescence photo bleaching recovery and correlation spectroscopy to measure the chemical kinetic binding rates and surface diffusion constant of fluorescent labeled serum protein binding to a surface at equilibrium.

Measured signals may be analyzed manually or by appropriate computer methods to tabulate results. The substrates and reaction conditions may include appropriate controls for verifying the integrity of hybridization and extension conditions, and for providing standard curves for quantification, if desired. For example, a control primer may be added to the polynucleotide sample for extending a target nucleic acid sequence that is known to be present in the sample or a target nucleic acid sequence that is added to the sample. The absence of the expected extension product is an indication that there is a defect with the sample or assay components requiring correction.

Practice of the invention will be still more fully understood from the following examples, which are presented herein for illustration only and should not be construed as limiting the invention in any way.

### EXEMPLIFICATION

### Example 1

Creation of Anchored Amplicons Using Rolling Circle Amplification

The creation of an anchored amplicon from a linear nucleic acid template using rolling circle amplification involves (i) a circularization reaction in which the 5′ and 3′ ends of a linear nucleic acid template are ligated to form a circular nucleic acid template; (ii) a hybridization reaction in which a primer is hybridized to a single stranded circular template to create a circular template-primer hybrid; and (iii) an extension reaction in which the primer is extended by rolling circle amplification. The primer may contain one member of a binding pair that can bind to a binding partner that is attached to a solid support.

Briefly, a nucleic acid template is obtained from a cell or tissue, for example, using one of a variety of procedures for extracting nucleic acids, which are well known in the art. While the invention is exemplified below with synthetic oligonucleotides, the invention is not so limited and may be practiced using any circular or circularized nucleic acids, including genomic DNA, cDNA, such as cDNA library, and RNA.

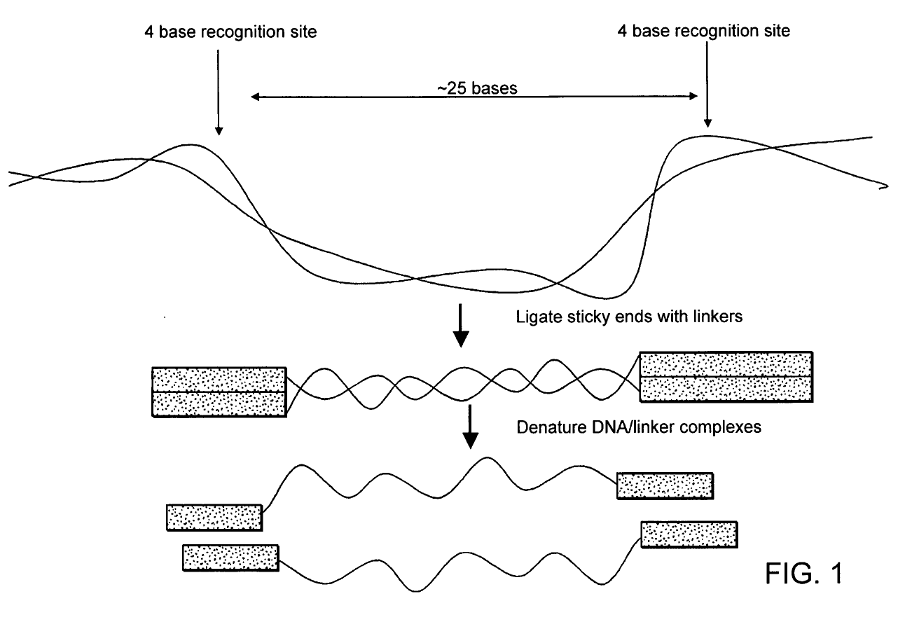

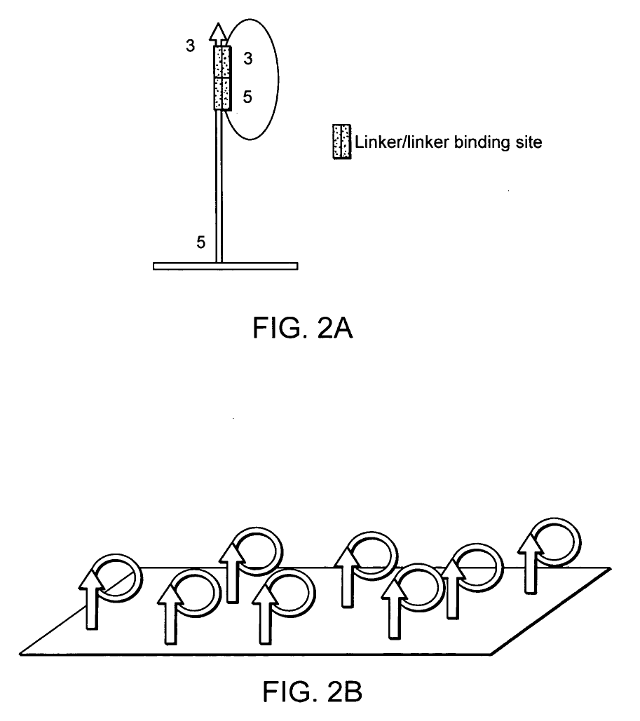

Nucleic acid that is linear is manipulated such that it can be circularized. Any known method of circularizing nucleic acids may be used to generate a circularized single-stranded nucleic acid template of the invention. For example, referring to FIG. 1, genomic DNA is digested with a frequent cleaving restriction enzyme to yield fragments of about 2 to about 1000 base pairs with a 5′ or 3′ overhang. Restriction enzymes useful in the invention include Bfa I, which cleaves C/TAG at 37° C., and Taq I, which cleaves T/CGA at 65°, both of which are sold by New England Biolabs (Beverly, Mass.). Linker nucleic acids are digested with the same restriction enzyme to yield compatible sticky ends and then ligated onto the 5′ and 3′ ends of the linear template fragments. To promote ligation, the concentration of the linkers is preferably low compared to the concentration of template molecules. Following denaturation of the double stranded DNA/linker complexes, the complexes are annealed to a single-stranded primer containing regions that are complementary to the linker sequences (FIG. 2A). The primer may be in solution or attached to a substrate during the annealing step. The primer anneals to the 5′ and 3′ linker sequences and the 5′ and 3′ ends of the DNA linker complexes are ligated together using double stranded DNA ligase such as T4 DNA ligase to create a circularized single stranded nucleic acid template-primer hybrid, as demonstrated in FIG. 2A.

Alternatively, a single stranded DNA ligase, such as CircLigase™ (Epicentre Biotechnologies, Madison, Wis.), may be used to circularize a linear single stranded nucleic acid template,. CircLigase™ is a thermostable ATP-dependent ligase that catalyzes intramolecular ligation (i.e., circularization) of single-stranded DNA (ssDNA) templates having a 5′-phosphate and a 3′-hydroxyl group in the absence of a complementary sequence. In this embodiment, linkers are not required and the anchor primer has a region that is complementary to sequence at the 5′ and 3′ ends of the linear template. The below example provides methods for generating a rolling circle amplification amplicon beginning with a single stranded nucleic acid template that is circularized using CircLigase™.

Circularization Reaction

Single stranded oligonucleotides of different lengths (33, 53, 66, 93, and 123 bases) were obtained and purified according to art known methods. Each circularization reaction contained 10 pmol single-stranded DNA, 1 μl 50 mM MnCl₂ (Epicentre Biotechnologies, Madison, Wis.), 1 μl 1 mM ATP (Epicentre Biotechnologies), 200 U CircLigase™ (Epicentre, cat no. CL4115K), and water to 20 μl. The circularization reaction was incubated at 61° C. for 1 hour and the enzyme was inactivated by incubation at 80° C. for 30 minutes. Circularization using CircLigase™ is most efficient at temperatures ranging from 6° C. to 69° C., with the best efficiency observed between 60° C. and 66° C.

All or a portion of the above circularization reaction was digested with Exo I, a 3′→5′ exonuclease that digests non-circularized single stranded DNA, to determine whether the linear single stranded nucleic acid templates had been circularized. An appropriate amount of 10× Exo I buffer (New England Biolabs, Beverly, Mass.) was add to make the concentration of Exo I buffer 1× and 20 U per 10 μl of Exo I (New England Biolabs, cat no. M0293) was added. The Exo I digestion reaction was incubated at 37° C. for 30 minutes and the enzyme was inactivated by incubation at 80° C. for 20 minutes. The digestion products were visualized on a small vertical TBE-urea gel.

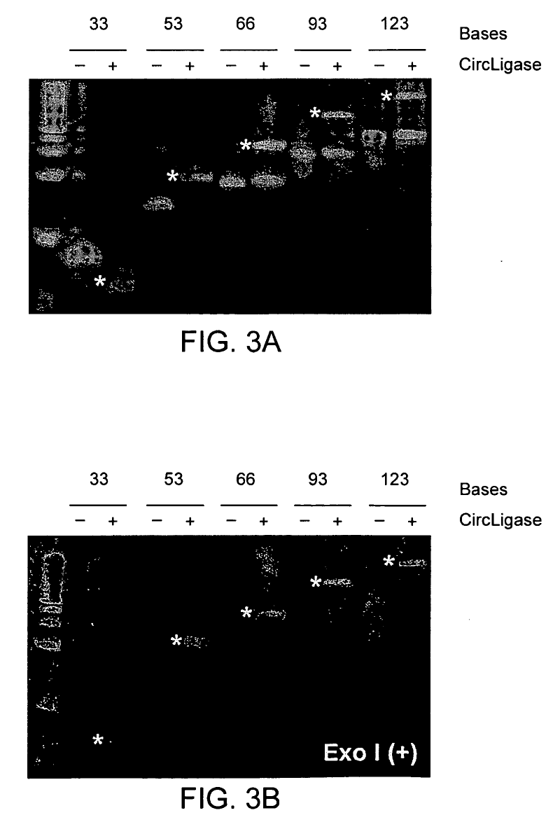

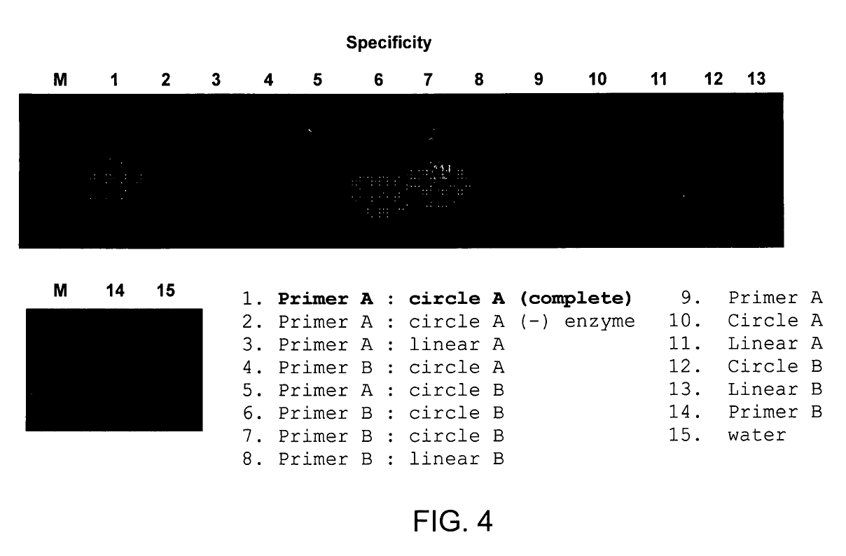

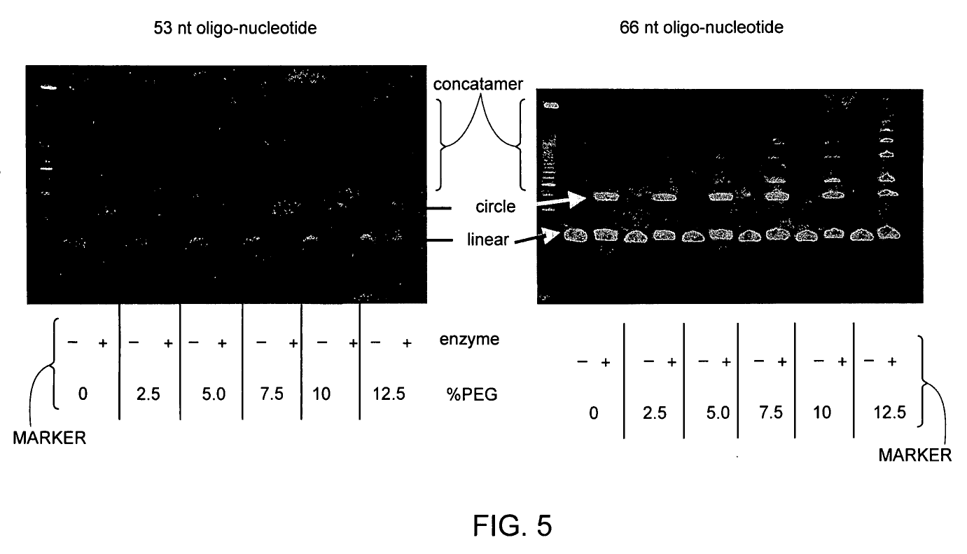

The results shown in FIG. 3A suggest that the length of the nucleic acid template affects the efficiency of circularization: the longer the nucleic acid template the lower the ratio of circular to linear molecules obtained in the reaction. For example, most of the 33 and 53 base oligonucleotides were circularized in the CircLigase™ reactions, with no lower band representing linear oligonucleotide apparent. However, the appearance of two bands in the CircLigase™ containing reactions for the 66, 93, and 123 base oligonucleotides indicates the presence of circular molecules (upper bands), as well as linear molecules (lower bands) that did not circularize. FIG. 4B shows that the lower bands in the CircLigase™ treated lanes for the 66, 93, and 123 oligonucleotides were eliminated by Exo I digestion, suggesting that those bands represented non-circularized templates.

Changes in the composition of the reaction buffer may also promote end-to-end ligation instead of circularization. For example, addition of PEG to the ligation reaction tends to cause end-to-end ligation instead of circularization.

Hybridization of a Primer to the Circular Template for Rolling Circle Amplification (RCA)

A primer is hybridized to the 5′ and 3′ end portions of the nucleic acid template. If the 5′ and 3′ ends of the linear template comprise linker DNA, the primer hybridizes to that linker sequence. In this embodiment, the primer has a biotin moiety at its 5′ end so that the primer can be attached to a streptavidin-coated surface. Each hybridization reaction contained 25 pmoles primer, 2.5 pmoles circular template, 1 μl 10× LSB buffer (100 mM Tris, pH 8.0, 1 M NaCl), and water to 10 μl in a 0.5 μl eppendorf tube. The tubes were incubated at 95° C. for 2 minutes, 40° C. for 10 minutes, and 20° C. for at least 10 minutes, using a PTC-200 Thermocycler (MJ Research) and cooled on ice.

Attachment of Circlular Template-Primer Hybrid to Streptavidin Tubes.

The above circular template-primer hybrid was attached to a streptavidin-coated tube (Roche, cat no. 1 741 772) to anchor the circular template-primer hybrid (FIGS. 2A and 2B). Each attachment reaction contained 2.0 μl circular template-primer hybrid DNA, 5.0 μl 10× HBS (100 mM Tris, pH 8.0, 3M NaCl), and 43.0 μl water. The reaction was incubated at 37° C. for 1 hour with constant shaking followed by 4° C. for at least one minute. The reaction mixture was then transferred to a fresh regular tube (i.e., not coated with a binding partner) and used to test the efficiency of the template-primer-streptavidin binding reaction (data not shown). The template-primer-streptavidin bound tubes were washed twice with ice-cold 1× polymerase buffer (made fresh by diluting 10× stock) (New England Biolabs, Beverly, Mass.).

Rolling Circle Amplification

The present invention contemplates limiting the rolling circle amplification reaction as it is traditionally conducted in order to exploit the low replication error rate of the reaction and to generate a limited number of copies of the nucleic acid template. In this example, rolling circle amplification is conducted on an primer-anchored circular template. The primer-anchored circular template is exposed to effective amounts of nucleotides, polymerase enzyme, and enzyme buffer. Nucleotide concentration is discussed below. An effective amount of polymerase may comprise about 10 nM to about 150 nM of φ29 polymerase, for example. The φ29 polymerase extends the anchored primer under isothermal conditions to create a linear amplicon of multiple complementary copies of the circular template. Preferred amplification temperatures are between about 20° C. and about 90° C., or between about 20° C. and about 50° C. For thermophylic enzymes, a preferred temperature for the reaction is between about 50° C. and about 100° C. By virtue of the anchored primer being attached to the substrate, the amplicon, which is an extension of the anchored primer, is attached to the substrate. See FIG. 6.

Methods of the invention provide for limiting the length of the concatamer complement formed by rolling circle amplification. The concentration of nucleotides is calculated such that a maximum of 50 complements of a nucleic acid template are created during the reaction. Preferably, depletion of nucleotides after several cycles of amplification limits the kinetics of the polymerization reaction, and ultimately, fewer than 50 complements are generated. The incorporation efficiency of the polymerase decreases as the available nucleotides become scarce. The reaction is arrested after a predetermined amount of time by washing away the remaining amplification reagents.

An exemplary nucleotide concentration calculation is as follows. The size of the genome is approximately 3×10⁹ bases. A sample comprises a digested genome, resulting in fragments of approximately 25 bases each, totaling approximately 1.2×10⁸ templates. Each template has a sequence comprising approximately 7 each of G, A, T, and C. To calculate the total of each nucleotide required to create amplicons equal to 50× complements of original template: (50)(7)(1.2×10⁸)=4.2×10¹⁰ each of G, A, T, and C=6.98×10⁻¹⁴ moles=0.07 picomoles of each nucleotide.

For rolling circle amplification using the above prepared template-primer-streptavidin tubes, the following components were added to each tube: 30 U φ29 polymerase enzyme (New England Biolabs, cat. no. M0269), 2.5 μl 10 mM dNTPs (Invitrogen), 5.0 μl 10× polymerase buffer (New England Biolabs), 0.5 μl of 100× bovine serum albumin (BSA) (New England Biolabs), and water to 50 μl. The tubes were incubated at 30° C. for a period of time that depended upon the degree of concatamerization desired, ranging from about 5 minutes to about 16 hours. Once the reaction was complete, the reaction was either stored, the nucleic acid was sequenced, or the amplified product was detached from the tube.

FIG. 4 shows the specificity of the rolling circle amplification reaction. Only those reactions containing a primer that is complementary to its circular template (e.g., primer A+template A or primer B+template B), in the presence of polymerase enzyme, resulted in an amplification product (Lanes 2 and 8).

FIG. 6 shows the results of rolling circle amplification reactions using either a 53 base oligonucleotide or a 66 base oligonucleotide, in the presence (+) or absence (−) of CircLigase™ enzyme, and in the presence or absence of various amounts of polyethylene glycol (PEG). The results demonstrate that the presence of 2.5, 5.0, 7.5, 10, or 12.5% PEG in the reaction mixture increases the number of concatamers of the template DNA in the reaction product. Treatment of the amplification products with Exo I digested the concatamers, suggesting that the rolling circle amplification amplified DNA was linear (data not shown).

### Example 2

Sequencing an Amplicon

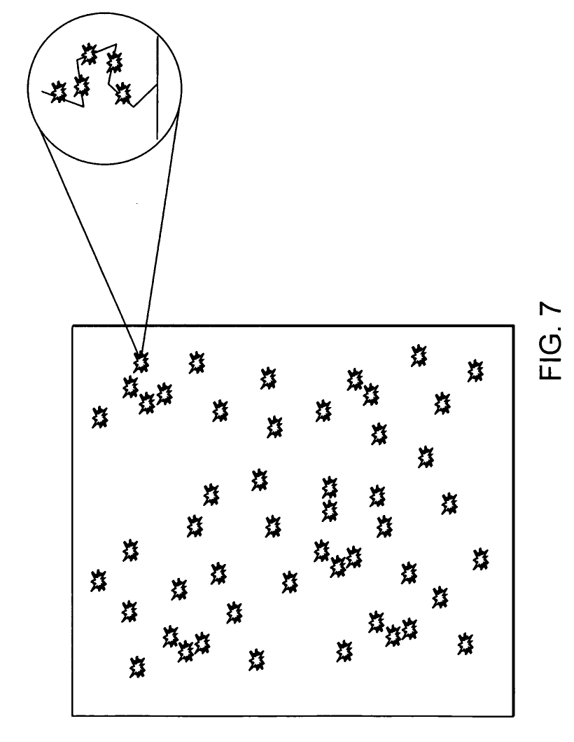

This example demonstrates a method according to the invention in which a single nucleotide in a position in a nucleic acid molecule is identified. At least one sequencing primer is bound to an amplicon. The sequence of the primer in this example complementary to the 3′ linker binding site on the anchored primer, or, in effect, identical to at least a portion of the 3′ linker sequence. Alternatively, if linkers are not used, the primer may be complementary to any region of the circular template, preferably the 3′ end. The amplicon/primer complex is exposed first to a labeled nucleotide and then to an unlabeled nucleotide of the same type under conditions of, and in the presence of, reagents that allow template-dependent primer extension (FIG. 6). The signals of the labeled amplicons are then detected (FIG. 7).

Cycle Sequencing of Rolling Circle Products Bound to Streptavidin Tubes

After the primer bound rolling circle amplification described in Example 1, the supernatant in the tubes was transferred to a fresh regular eppendorf tube (i.e., that did not contain bound streptavidin). The supernatant can be tested for the presence of rolling circle amplification product that is not bound to the tube (data not shown). The primer-RCA-streptavidin bound tube was washed once with 80 μl Tris B (10 mM Tris, pH 8.0, 10 mM NaCl) and once with 50 μl 10× BigDyeg buffer (Applied BioSystems, Foster City, Calif.). The following components were then added to each tube: 5 pmoles of sequencing primer (5′ TTCCACCTTCTCCAAGAACTATAT 3′, 4 μl of 5× BigDye® buffer (Applied BioSystems), 8 μl of BigDye® (Applied BioSystems), and water to 20 μl. The sequencing reactions took place under the following conditions using a PTC-200 thermocycler: 95° C. for 1 minute; 28×[95° C. for 10 seconds; 50° C. for 5 seconds; 60° C. for 2 minutes]; 60° C. for 5 minutes; hold at 4° C.

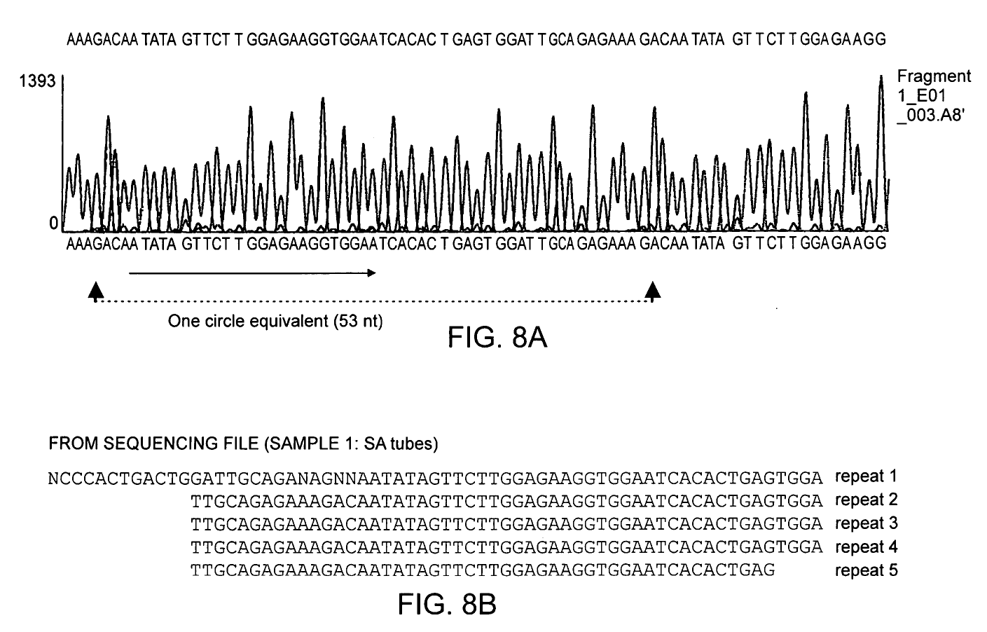

FIG. 8A is an electropherogram of the sequencing reaction generated using a ABI Prism 3700 DNA Sequence Analyser (Applied BioSystems). FIG. 8B shows the sequence of RCA-amplified product and confirms the presence of multiple repeats of the predicted circular template sequence. The sequence to which the primer binds is bold.

Detaching the Rolling Circle Amplification Products

After the sequencing reaction of the primer bound rolling circle amplification described above, the supernatant in the tubes was transferred to a fresh eppendorf tube that did not contain bound streptavidin. The supernatant was tested to assess the sequencing reaction (data not shown). The primer-RCA-streptavidin bound tube was washed twice with 80 μl of Tris B (10 mM Tris, pH 8.0; 10 mM NaCl). The following components were then added to the tube: 50 μl of 10 mM EDTA, 95% deionized formamide (Applied Biosystems) for at 65° C. for 8 minutes.

### Example 3

Analysis of Single Molecule Sequencing

Using a TIR Optical Setup such as that diagrammed in FIG. 9, images of a surface on which single molecule sequencing of an attached rolling circle amplified template has been performed are then analyzed for primer-incorporated U-Cy5. Typically, eight exposures of 0.5 seconds each are taken in each field of view in order to compensate for possible intermittency (e.g., blinking) in fluorophore emission. Software is employed to analyze the locations and intensities of fluorescence objects in the intensified charge-coupled device pictures. Fluorescent images acquired in the WinView32 interface (Roper Scientific, Princeton, N.J.) are analyzed using ImagePro Plus software (Media Cybernetics, Silver Springs, Md.). Essentially, the software is programmed to perform spot-finding in a predefined image field using user-defined size and intensity filters. The program then assigns grid coordinates to each identified spot, and normalizes the intensity of spot fluorescence with respect to background across multiple image frames. From those data, specific incorporated nucleotides are identified. Generally, the type of image analysis software employed to analyze fluorescent images is immaterial as long as it is capable of being programmed to discriminate a desired signal over background. The programming of commercial software packages for specific image analysis tasks is known to those of ordinary skill in the art. If U-Cy5 is not incorporated, the substrate is washed, and the process is repeated with dGTP-Cy5, dATP-Cy5, and dCTP-Cy5 until incorporation is observed. The label attached to any incorporated nucleotide is neutralized, and the process is repeated. To reduce bleaching of the fluorescence dyes, an oxygen scavenging system may be used during all green illumination periods, with the exception of the bleaching of the primer tag.

The template is analyzed in order to determine whether the first nucleotide is incorporated in any of the plurality of bound primers at the first position. No detectable signal indicates that the first nucleotide was not incorporated, so that the sequential exposure to labeled and unlabeled nucleotides is repeated using another type of nucleotide until one such nucleotide is determined to have incorporated at the first position. Once an incorporated nucleotide is detected, the nucleotide in that position in the nucleic acid template sequence is identified.

In this example, during the addition of each nucleotide an incorporation event may occur at multiple identical loci on an amplicon. See FIG. 6. The unlabeled nucleotide may “fill in” positions on complements in the amplicon that do not incorporate a labeled nucleotide. Signal from incorporated labeled nucleotides is detectable, however, and may be about 2 to about 100 times greater than signal on a single copy template, or on an unamplified nucleic acid template. Furthermore, incorporation of unlabeled nucleotides in a subset of template complements may encourage incorporation of labeled nucleotides during the subsequent addition step due to lower steric hindrance than incorporating multiple labeled nucleotides in a row.

### Incorporation by Reference

The contents of all cited references (including literature references, patents, and patent applications) that may be cited throughout this application are hereby expressly incorporated by reference. The practice of the present invention will employ, unless otherwise indicated, conventional techniques of nucleic acid preparation, manipulation, and sequencing, which are well known in the art.

### Equivalents

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

We claim:

1. A method of determining a sequence of a nucleic acid, the method comprising the steps of:
(a) conducting rolling circle amplification of a nucleic acid to produce an amplicon comprising about two to about one hundred linked complements of a nucleic acid; wherein said amplicon is anchored to a substrate, such that said amplicon is individually optically resolvable; and
(b) determining a sequence of at least a portion of said nucleic acid.

2. The method of claim 1, wherein said nucleic acid is genomic DNA, cDNA, or RNA.

3. The method of claim 1, wherein said amplification step comprises circularizing said nucleic acid thereby forming a circular template; combining said circular template with a primer, a polymerizing agent, and nucleotides; and producing an amplicon comprising multiple linked complements of said circular template.

4. The method of claim 3, wherein said primer binds to at least one of the 3′ end and the 5′ end of the nucleic acid.

5. The method of claim 1, wherein said nucleic acid is single stranded.

6. The method of claim 1, wherein said nucleic acid is double stranded, and wherein said amplification step further includes the step of denaturing said double stranded nucleic acid prior to combining with said primer.

7. The method of claim 1, wherein said amplicon is anchored to said substrate after completion of said amplification step.

8. The method of claim 1, wherein said amplicon is covalently bound to said substrate.

9. The method of claim 1, wherein said amplicon is anchored to said substrate via a biotin-streptavidin complex.

10. The method of claim 1, wherein said amplicon comprises a number of linked complements of the nucleic acid, said number determined by a concentration of nucleotides available for incorporation into said amplicon.

11. The method of claim 1, wherein said substrate comprises an accumulation of negative charge.

12. The method of claim 1, wherein said substrate comprises a plurality of loci for anchoring said amplicon.

13. The method of claim 1, wherein said substrate is selected from the group consisting of glass, fused silica, epoxy, plastic, metal, gel matrix, and composites.

14. The method of claim 13, wherein said substrate has a chemically modified surface comprising a polyelectrolyte multilayer.

15. The method of claim 1, wherein said determining step comprises exposing said amplicon to a sequencing primer, a polymerizing agent, and at least one nucleotide; allowing incorporation of said nucleotide(s) into a synthesis strand; detecting incorporation of said nucleotide(s); and repeating said determining step at least once, thereby determining said sequence of said nucleic acid.

16. The method of claim 15, wherein said determining step results in incorporation of about one or about two nucleotides into said synthesis strand.

17. The method of claim 15, wherein said nucleotide is labeled with a fluorescent moiety.

18. The method of claim 1, wherein said amplification step is performed with a primer that is anchored to said substrate such that upon completion of said amplification step, said amplicon is anchored to said substrate.

19. The method of claim 1, wherein said amplification step is performed with a primer that is not anchored to said substrate such that upon completion of said amplification step, said amplicon is not attached to said substrate.


SEQUENCE LISTING (5 sequences)

SEQ ID NO: 1 - DNA, 24 bp, Artificial Sequence
  Description of Artificial Sequence Synthetic primer
ttccaccttctccaagaactatat

SEQ ID NO: 2 - DNA, 79 bp, Artificial Sequence
  Description of Artificial Sequence Synthetic oligonucleotide
aaagacaatatagttcttggagaaggtggaatcacactgagtggattgcagagaaagacaatatagttcttggagaagg

SEQ ID NO: 3 - DNA, 65 bp, Artificial Sequence
  Description of Artificial Sequence Synthetic oligonucleotide
ncccactgactggattgcaganagnnaatatagttcttggagaaggtggaatcacactgagtgga

SEQ ID NO: 4 - DNA, 53 bp, Artificial Sequence
  Description of Artificial Sequence Synthetic oligonucleotide
ttgcagagaaagacaatatagttcttggagaaggtggaatcacactgagtgga

SEQ ID NO: 5 - DNA, 49 bp, Artificial Sequence
  Description of Artificial Sequence Synthetic oligonucleotide
ttgcagagaaagacaatatagttcttggagaaggtggaatcacactgag